(19) United States
(12) Patent Application Publication
(10) Pub. No.: US 2024/0285555 A1
(43) Pub. Date: Aug. 29, 2024

(54) COMPOSITIONS FOR THE TREATMENT OF COPPER DEFICIENCY AND METHODS OF USE

(71) Applicant: The Texas A&M University System, College Station, TX (US)

(72) Inventors: Shivatheja Soma, College Station, TX (US); Vishal M. Gohil, College Station, TX (US); James C. Sacchettini, College Station, TX (US); Liam Guthrie, College Station, TX (US)

(73) Assignee: The Texas A&M University System, College Station, TX (US)

(21) Appl. No.: 18/652,683

(22) Filed: May 1, 2024

Related U.S. Application Data
(62) Division of application No. 17/257,770, filed on Jan. 4, 2021.
(60) Provisional application No. 62/697,207, filed on Jul. 12, 2018.

(51) Int. Cl. A61K 31/165; A61K 33/34; A61P 3/02

(52) U.S. Cl. CPC A61K 31/165; A61K 33/34; A61P 3/02

(57) ABSTRACT

In an embodiment, the present disclosure relates to a method of restoring cytochrome c oxidase (CcO) activity in a subject in need thereof. In some embodiments, the method includes administering a therapeutically effective amount of elesclomol or analog thereof and rescuing defects of cells in the subject with deficiencies or mutations in at least one of SOD1, AT-1, APIS1, COA6, SCO2, COX6B1, CTR1, ATOX1, CCS, GSX1, ATP7A, ATP7B, CLCN5, and CLCN7. In a further embodiment, the present disclosure relates to a method of treating disorders of copper metabolism. In some embodiments, the method includes administering a therapeutically effective amount of elesclomol or analog to a subject, where the disorder is caused by a deficiency or mutation to a gene including, without limitation, SOD1, AT-1, APIS1, COA6, SCO2, COX6B1, CTR1, ATOX1, CCS, GSX1, ATP7A, ATP7B, CLCN5, CLCN7, or combinations thereof.

### CROSS-REFERENCE TO RELATED APPLICATIONS

This application is a divisional application of U.S. Ser. No. 17/257,770 filed Jan. 4, 2021, which is a US National Stage filing of PCT/US2019/041571 filed Jul. 12, 2019, which claims priority from, and incorporates by reference the entire disclosure of, U.S. Provisional Application No. 62/697,207 filed on Jul. 12, 2018.

### STATEMENT REGARDING FEDERALLY SPONSORED RESEARCH

This invention was made with government support under GM111672 awarded by the National Institutes of Health. The government has certain rights in the invention.

### REFERENCE TO AN ELECTRONIC SEQUENCE LISTING

The contents of the electronic sequence listing (13260P058WUSD1_Seq.xml; Size: 10,936 bytes; and Date of Creation: May 1, 2024) is herein incorporated by reference in its entirety.

### TECHNICAL FIELD

The present disclosure relates generally to copper deficiency and more particularly, but not by way of limitation, to compositions for the treatment of copper deficiency and methods of use.

### BACKGROUND

This section provides background information to facilitate a better understanding of the various aspects of the disclosure. It should be understood that the statements in this section of this document are to be read in this light, and not as admissions of prior art.

Copper is an essential cofactor of cytochrome c oxidase (CcO), the terminal enzyme of the mitochondrial respiratory chain. Inherited loss-of-function mutations in several genes encoding proteins required for copper delivery to CcO result in diminished CcO activity and severe pathology in affected infants. Copper supplementation restores CcO function in patient cells with mutations in two of these genes, COA6 and SCO2, suggesting a potential therapeutic approach. However, direct copper supplementation has not been therapeutically effective in human patients, underscoring the need to identify highly efficient copper transporting pharmacological agents. Utilizing a candidate-based approach, an investigational anti-cancer drug, elesclomol (ES), that rescues respiratory defects of COA6 deficient yeast cells by increasing mitochondrial copper content and restoring CcO activity was identified. ES also rescues respiratory defects in other yeast mutants of copper metabolism, suggesting broader applicability. Low nanomolar concentrations of ES reinstate copper-containing subunits of CcO in a zebrafish model of copper deficiency and in a series of copper deficient mammalian cells, including those derived from a SCO2 patient. The findings presented herein reveal that ES can restore intracellular copper homeostasis by mimicking the function of missing transporters and chaperones of copper, and may have potential in treating human disorders of copper metabolism.

Inherited pathogenic mutations in genes required for copper delivery to cytochrome c oxidase (CcO) perturb mitochondrial energy metabolism and result in fatal mitochondrial disease. A prior attempt to treat human patients with these mutations by direct copper supplementation was not successful, possibly because of inefficient copper delivery to the mitochondria. A targeted search was performed to identify compounds that can efficiently transport copper across biological membranes and elesclomol, an investigational anti-cancer drug, was identified as the most efficient copper delivery agent. Elesclomol rescues CcO function in yeast, zebrafish, and mammalian models of copper deficiency by increasing cellular and mitochondrial copper content. Thus, the present disclosure offers a possibility of repurposing this anti-cancer drug for the treatment of disorders of copper metabolism.

The development of this invention was funded in part by the Welch Foundation under grant number A-1810.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In some embodiments, the present disclosure relates to a method of restoring cytochrome c oxidase (CcO) activity in a subject in need thereof. In some embodiments, the method includes administering a therapeutically effective amount of elesclomol, and rescuing defects of cells in the subject with deficiencies or mutations in at least one of SOD1, AT-1, AP1S1/COA6, SCO2, COX6B1, CTR1, ATOX1, CCS, GSX1, ATP7A, ATP7B, CLCN5, and CLCN7. In some embodiments, the administering increases at least one of cellular copper content and mitochondrial copper content. In some embodiments, the administering reestablishes subcellular copper homeostasis in copper deficient cells. In some embodiments, the administering ameliorates defects of at least one of cellular copper homeostasis and mitochondrial copper homeostasis.

In some embodiments, the method further includes mimicking functions of missing transporters or chaperones of copper and restoring intracellular copper homeostasis. In some embodiments, the method additionally includes transporting copper across biological membranes and restoring mitochondrial respiratory chain function. In some embodiments, the therapeutically effective amount of elesclomol for a human subject is in a range of about 0.589 mg/kg body weight. In some embodiments, the therapeutic dosage range for elesclomol in humans is 0.243-1.17 mg/kg. In some embodiments, the elesclomol is an elesclomol analog, mimetic, or derivatives thereof. In some embodiments, the method further includes bypassing at least one of SCO2 functions and COA6 functions.

In a further embodiment, the present disclosure relates to a method of treating disorders of copper metabolism. In some embodiments, the method includes administering a therapeutically effective amount of elesclomol to a subject, where the disorder is caused by a deficiency or mutation to a gene including, without limitation, SOD1, AT-1, AP1S1/COA6, SCO2, COX6B1, CTR1, ATOX1, CCS, GSX1, ATP7A, ATP7B, CLCN5, CLCN7, or combinations thereof. In some embodiments, the disorder is caused by a mutation to the ATP7A gene. In some embodiments, the disorder can include, without limitation, occipital horn syndrome, X-linked distal hereditary motor neuropathy, amyotrophic lateral sclerosis, Lou Gehrig disease, Alzheimer's disease, Huppke-Brendel syndrome, MEDNIK syndrome, or combinations thereof. In some embodiments, the administering increases at least one of cellular copper content and mitochondrial copper content. In some embodiments, the administering reestablishes subcellular copper homeostasis in copper deficient cells. In some embodiments, the administering ameliorates defects of at least one of cellular copper homeostasis and mitochondrial copper homeostasis.

In some embodiments, the method further includes mimicking functions of missing transporters or chaperones of copper and restoring intracellular copper homeostasis. In some embodiments, the method additionally includes transporting copper across biological membranes and restoring mitochondrial respiratory chain function. In some embodiments, the method includes co-administering elesclomol and copper to a subject. In some embodiments, the therapeutically effective amount is in a range of about 0.589 mg/kg body weight. In some embodiments, the therapeutic dosage range for elesclomol in humans is 0.243-1.17 mg/kg.

In some embodiments, the elesclomol is elesclomol complexed with copper (Cu(II)-ES), an elesclomol analog, an elesclomol mimetic, or derivatives thereof. In some embodiments, the method further includes bypassing at least one of SCO2 functions and COA6 functions.

In a further embodiment, the present disclosure relates to a compound having a structure as represented in FIG. 11B. In some embodiments, X₁, X₂, X₃, and X₄ can each, independently, include, without limitation, O, S, Se, Te, Po, N(R₇)ₘ, P(R₇)ₘ, As(R₇)ₘ, Sb(R₇)ₘ, or Bi(R₇)ₘ, or combinations thereof. In some embodiments, m is 0 or 1. In some embodiments, R₁, R₂, R₃, R₄, R₅, and R₆ can each, independently, include, without limitation, —H, an alkyl, an alkenyl, an alkynyl, a cycloalkyl, a cycloalkenyl, a heterocyclyl, an aryl, a heteroaryl, a halogen, a nitro, a cyano, a guanadino, —OR₈, —NR₁₀R₁₁, —C(O)R₈, —C(O)OR₈, —OC(O)R₈, —C(O)NR₁₀R₁₁, —NR₉C(O)R₈, —OP(O)(OR₈)₂, —SP(O)(OR₈)₂, —SR₈, —S(O)ₚR₈, —OS(O)ₚR₈, —S(O)ₚOR₈, —NR₉S(O)ₚR₈, —S(O)ₚNR₁₀R₁₁, an aromatic, or combinations thereof. In some embodiments, R₇ can include, without limitation, —H, —OR₈, —NR₁₀R₁₁, —C(O)R₈, —C(O)OR₈, —OC(O)R₈, —C(O)NR₁₀R₁₁, —NR₉C(O)R₈, —OP(O)(OR₈)₂, —SP(O)(OR₈)₂, —SR₈, —S(O)ₚR₈, —OS(O)ₚR₈, —S(O)ₚOR₈, —NR₉S(O)ₚR₈, —S(O)ₚNR₁₀R₁₁, an alkyl, an alkenyl, an alkynyl, an cycloalkyl, an cycloalkenyl, an heterocyclyl, an aryl, a heteroaryl, an aralkyl, a heteraralkyl, a halogen, a nitro, a cyano, a guanadino, an aromatic, or combinations thereof. In some embodiments, p is 1 or 2. In some embodiments, R₈, R₉, R₁₀, and R₁₁ can each, independently, include, without limitation, —H, an alkyl, an alkenyl, an alkynyl, a cycloalkyl, a cycloalkenyl, a heterocyclyl, an aryl, a heteroaryl, an aralkyl, a heteraralkyl, a halogen, a nitro, a cyano, a guanadino, an aromatic, or combinations thereof. In some embodiments, at least one of R₁₀ and R₁₁ are taken together with the nitrogen to which they are attached to form a heterocyclyl or a heteroaryl.

In another embodiment, the present disclosure relates to a pharmaceutical composition having a structure as represented in FIG. 11B, or a tautomer, pharmaceutically acceptable salt, solvate, clathrate, or prodrug thereof. In some embodiments, the composition further includes an excipient that can include, without limitation, salts, solvents, buffers, diluents, binders, compression aids, granulating agents, disintegrants, glidants, lubricants, tablet coatings, tablet films, coloring agents, or combinations thereof. In some embodiments, X₁, X₂, X₃, and X₄ can each, independently, include, without limitation, O, S, Se, Te, Po, N(R₇)ₘ, P(R₇)ₘ, As(R₇)ₘ, Sb(R₇)ₘ, or Bi(R₇)ₘ, or combinations thereof. In some embodiments, m is 0 or 1. In some embodiments, R₁, R₂, R₃, R₄, R₅, and R₆ can each, independently, include, without limitation, —H, an alkyl, an alkenyl, an alkynyl, a cycloalkyl, a cycloalkenyl, a heterocyclyl, an aryl, a heteroaryl, a halogen, a nitro, a cyano, a guanadino, —OR₈, —NR₁₀R₁₁, —C(O)R₈, —C(O)OR₈, —OC(O)R₈, —C(O)NR₁₀R₁₁, —NR₉C(O)R₈, —OP(O)(OR₈)₂, —SP(O)(OR₈)₂, —SR₈, —S(O)ₚR₈, —OS(O)ₚR₈, —S(O)ₚOR₈, —NR₉S(O)ₚR₈, —S(O)ₚNR₁₀R₁₁, an aromatic, or combinations thereof. In some embodiments, R₇ can include, without limitation, —H, —OR₈, —NR₁₀R₁₁, —C(O)R₈, —C(O)OR₈, —OC(O)R₈, —C(O)NR₁₀R₁₁, —NR₉C(O)R₈, —OP(O)(OR₈)₂, —SP(O)(OR₈)₂, —SR₈, —S(O)ₚR₈, —OS(O)ₚR₈, —S(O)ₚOR₈, —NR₉S(O)ₚR₈, —S(O)ₚNR₁₀R₁₁, an alkyl, an alkenyl, an alkynyl, an cycloalkyl, an cycloalkenyl, an heterocyclyl, an aryl, a heteroaryl, an aralkyl, a heteraralkyl, a halogen, a nitro, a cyano, a guanadino, an aromatic, or combinations thereof. In some embodiments, p is 1 or 2. In some embodiments, R₈, R₉, R₁₀, and R₁₁ can each, independently, include, without limitation, —H, an alkyl, an alkenyl, an alkynyl, a cycloalkyl, a cycloalkenyl, a heterocyclyl, an aryl, a heteroaryl, an aralkyl, a heteraralkyl, a halogen, a nitro, a cyano, a guanadino, an aromatic, or combinations thereof. In some embodiments, at least one of R₁₀ and R₁₁ are taken together with the nitrogen to which they are attached to form a heterocyclyl or a heteroaryl.

An embodiment of the invention is directed to compounds for treating disorders of copper metabolism. In certain embodiments, the compounds of the invention are represented by the structures set out in FIG. 12.

### BRIEF DESCRIPTION OF THE DRAWINGS

A more complete understanding of the methods and compositions of the present disclosure may be obtained by reference to the following Detailed Description when taken in conjunction with the accompanying Drawings wherein:

FIG. 1 illustrates determination of the median effective dose (ED₅₀) of elesclomol. Yeast coa6Δ cells were cultured in YPGE medium at 37° C. in the presence of increasing concentrations (0.01 nM to 1 μM) of elesclomol (ES). The cell density was measured spectrophotometrically after 58 hours of growth at 600 nm. The data represent the average±SD from three independent measurements.

FIGS. 2A, 2B, 2C, 2D and 2E illustrate elesclomol supplementation rescues CcO assembly defects by restoring mitochondrial copper levels of yeast S. cerevisiae coa6Δ cells. FIGS. 2A-2C illustrate bioenergetic parameters of wild type (WT), coa6Δ, and coa6Δ cells supplemented with 20 nM ES were cultured in YP galactose medium until early stationary growth phase. FIG. 2A shows oxygen consumption rate (OCR) measurement. FIG. 2B shows quantification of supercomplexes. FIG. 2C shows quantification of CcO activity. FIG. 2D shows mitochondrial copper levels. FIG. 2E shows total cellular copper content. Error bars represent mean±SD (n=3, two-tailed unpaired Student's t-test *p<0.05, **p<0.005).

FIGS. 3A and 3B illustrates elesclomol rescue of respiratory growth of coa6Δ cells is dependent on copper availability. BY4741 WT, FIG. 3A, and coa6Δ, FIG. 3B, cells were cultured in YPGE medium at 37° C. in the presence of 10 nM ES, 20 μM of the copper chelator bathocuproine disulfonic acid (BCS), or a combination of both. The cell density was measured spectrophotoretrically at the indicated time points at 600 nm. The data are representative of two independent experiments.

FIGS. 4A and 4B illustrates the effects of ES supplementation in increasing copper levels (A) and cytochrome c oxidase subunit, COX1, and superoxide dismutase, SOD1 levels (B) in Ctrl1^{-/-} H9c2 rat cardiomyocytes. ATP5A is a subunit of mitochondrial ATP synthase and was used as a loading control for the Western blotting experiment.

FIGS. 5A and 5B illustrate cell viability of immortalized and primary human fibroblasts cell lines treated with increasing concentrations of elesclomol. FIG. 5A shows immortalized control (MCH46) and SCO2 patient fibroblasts. FIG. 5B shows primary human skin fibroblasts (MW28) cultured in the DMEM glucose-containing media were treated with increasing concentrations of ES for 36 hours. Cell viability was measured by quantifying total cellular ATP levels by Cell-Titre GLo assay (Promega). Data are expressed as mean±SD (n=3).

FIGS. 6A, 6B, 6C and 6D illustrate that elesclomol treatment improves morphological defects observed in Coa6 knockdown embryos. FIG. 6A shows zebrafish embryos were treated with indicated concentrations of ES. FIG. 6B shows zebrafish embryos were treated with indicated concentrations of ES, copper, and combinations of both at 3 hours post fertilization (hpf). The surviving fish were counted at the indicated time points (n=30 for each treatment). FIG. 6C shows phenotypic scores of untreated and ES treated Coa6 knockdown zebrafish embryos at the indicated time points (hpt). Batches of 50 fertilized embryos injected with either translation blocking or mismatch control morpholinos were treated with or without 100 nM ES at 3 hpf, before making observations at the indicated time points (n % 30 for each time point and treatment). FIG. 6D shows heart rate of zebrafish embryos injected with either mismatch control morpholino or the Coa6 translation blocking morpholino that were treated with ES at the indicated time point (n≥30 per group; data represent mean±SEM). NT, No treatment; ES, Elesclomol.

FIG. 7 illustrates two injections of elesclomol-Cu prevents early death of Menkes (mo-br) mice. Kaplan-Meier Survival of mice after subcutaneous injections of ES-Cu(II) on postnatal days 7 and 10. Log-Rank statistical analysis was conducted to assess significance of survival among mo-br mice cohorts. Mo-br mice treated with 3.625 mg/kg/dose. ES-Cu(II) experienced 81.5% 10 week survival rate with a mean survival time of 63.4±15.8 days compared to vehicle only 14.2±0.2, equivalent Cu²⁺ dose of Cu(II)-2Histidinate 18.2±1.1, and equivalent elesclomol only dose of 3.15 mg/kg/dose 17.0±0.9 days (P<0.0001).

FIG. 8 illustrates mean body weight gain of mice after subcutaneous injections on postnatal days 7 and 10. Cohorts and number of mice/cohort are designated as follows: WT vehicle (n=13), WT Cu(II)-ES (n=12), mo-br vehicle (n=9), mo-br Cu(II)-ES (n=27), mo-br Cu(II)-2His (n=6), and mo-br ES (n=6). Growth curves are reported as the mean of each cohort±SD measured weekly. No mo-br mice belonging to the vehicle or ES treatment cohorts survived past the week 2 time points (not shown). All mo-br Cu(II)-2His treated mice expired by week 3.

FIG. 9 illustrates inverted wire screen tests of muscle strength in surviving mice cohorts at 5, 10, 15, and 20 weeks assessment. Mice were placed in a 43 cm² mesh with 12 mm length squares of 1 mm diameter wire and inverted 180° for 60 seconds. Best of three trials separated by a rest period of fifteen minutes was recorded every five weeks during mid-light cycle. ANOVA with Dunnett's (two-sided) post hoc test was used to assess significance.

FIG. 10 illustrates motor performance assessed by accelerating rotarod in surviving mice cohorts at 10 weeks of age. Mice were trained on the rotarod (UGO Basile model 7650) for three sessions at constant rotation of 4 rpm before testing. To assess motor performance, mice were placed on an accelerating rotarod (4 rpm to 40 rpm 300 seconds followed by constant rotation 120 seconds) for a maximum of 420 seconds. Latency to fall or passive rotation was recorded and averaged for three trials per mouse. All mice were assessed during mid-light cycle in randomized order. Data is reported as the average cohort mean±SEM. ANOVA with Dunnett's post hoc test was used to determine significance.

FIG. 11A illustrates the chemical structure of elesclomol.

FIG. 11B illustrates a generic chemical structure for analogs of elesclomol according to aspects of the present disclosure.

FIG. 12 illustrates example analogs of elesclomol according to aspects of the present disclosure.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Copper is an essential micronutrient required for the assembly and activity of cytochrome c oxidase (CcO), the terminal enzyme of the mitochondrial respiratory chain that catalyzes the reduction of molecular oxygen and drives mitochondrial energy production. CcO is a highly conserved, multimeric inner mitochondrial membrane protein complex that has two copper-containing subunits, Cox1 and Cox2, which together form its catalytic core. Copper delivery to mitochondria and its insertion into these copper-containing subunits is an intricate process that requires multiple metallochaperones and ancillary proteins. Failure to deliver copper to Cox1 and Cox2 disrupts CcO assembly and results in a respiratory deficiency.

Cytosolic copper is delivered to the mitochondrial matrix via the recently identified yeast protein Pic2, where it is stored in a ligand bound form. This mitochondrial matrix copper pool is the main source of copper ions that are inserted into the CcO subunits in the mitochondrial inter-membrane space (IMS). Mobilization of copper from the mitochondrial matrix to the IMS for its delivery to copper sites in CcO subunits requires a number of evolutionarily conserved proteins. The precise molecular functions of these proteins have remained unsolved, except for the metallochaperones Cox17, Sco1, Sco2 and Cox 11, which have been shown to transfer copper to CcO subunits in a bucket-brigade fashion. Specifically, Cox17 receives copper from the mitochondrial matrix and transfers it to Cox11 and Sco1/Sco2, which then metallate copper sites on Cox1 and Cox2, respectively. Recently two other proteins, Coa6 and Cox19, have also been shown to be part of this copper delivery pathway in the IMS.

In humans, inherited partial loss-of-function mutations in SCO1, SCO2, and COA6 result in a CcO deficiency and are associated with hepatopathy, metabolic acidosis, cardiomyopathy, and neurological defects in affected patients. Copper supplementation rescues CcO deficiency in myoblasts from patients with mutations in SCO2 and restores CcO activity in COA6 deficient yeast and human patient cell lines, suggesting that efficient delivery of copper to mitochondria could restore CcO activity by bypassing SCO2 and COA6 functions. In an attempt to translate these observations in a clinical setting, subcutaneous injections of copper-histidine were administered to a patient with a SCO2 mutation. While copper supplementation improved the patient's hypertrophic cardiomyopathy, it did not improve other clinical outcomes or survival. Thus, a more effective mechanism for restoration of copper homeostasis will be required for human therapeutics. The present disclosure employed yeast coa6Δ cells to identify compounds that can efficiently transport copper across biological membranes and restore mitochondrial respiratory chain function over a broad range of concentrations. This approach identified elesclomol (ES), which was shown to reestablish subcellular copper homeostasis in copper deficient cells, highlighting its therapeutic potential for human diseases of copper metabolism.

ATP7A is an ATP-driven copper transport protein that plays an essential role in human health. ATP7A is critically involved in dietary copper uptake from the intestine. In addition, ATP7A delivers copper to numerous copper-dependent enzymes within the secretory pathway and facilitates copper transfer to the brain. Inactivating mutations in ATP7A are associated with severe and often lethal pathologies, such as Menkes disease, occipital horn syndrome, and X-linked distal hereditary motor neuropathy. Genetic and biochemical studies have demonstrated that disease-causing mutations disrupt ATP7A in many ways, including disruption of biosynthesis, impairment of stability, inactivation of copper transport activity, and ATP7A trafficking. Elesclomol is a viable therapeutic for the treatment of copper metabolism disorders caused by mutations in ATP7A.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

Reagents. All the copper binding compounds were purchased from Sigma-Aldrich, except Elesclomol (ES), which was purchased from Selleckchem. The yeast-human hybrid (hyCOA6) gene construct was codon optimized for yeast and synthesized using GENEART® Gene Synthesis (Life Technologies). The hybrid gene hyCOA6 was cloned into pRS416 plasmid under the control of the yeast Coa6 native promoter. COA6 patient mutations were introduced by site-directed-mutagenesis (Agilent Technologies QuikChange Lightning) using hyCOA6 as a template. All the primers used in the present disclosure are listed in Table 1, shown below. All the constructs were sequenced verified.

TABLE 1

| Name | Sequence (5′→3′) | |
|---|---|---|
| Site Directed Mutagenesis | | |
| Primers | | |
| HyCOA6 W26C Forward | agtattcatctctagcaccgcaacacaattttctttggg | (SEQ ID NO: 1) |
| HyCOA6 W26C Reverse | cccaaagaaaattgtgttgcggtgctagagatgaatact | (SEQ ID NO: 2) |
| HyCOA6 W33R Forward | ttttcgtccaaacatttccggtattcatctctagcaccc | (SEQ ID NO: 3) |
| HyCOA6 W33R Reverse | gggtgctagagatgaataccggaaatgtttggacgaaaa | (SEQ ID NO: 4) |
| HyCOA6 E54X Forward | cattgttgtggacaagaagattagaaagaggatctcaacttctta | (SEQ ID NO: 5) |
| HyCOA6 E54X Reverse | taagaagttgagatcctctttctaatcttcttgtccacaacaatg | (SEQ ID NO: 6) |

Yeast Strains and Culture Conditions. Saccharomyces cerevisiae strains used in the present disclosure are listed in Table 2, shown below. The authenticity of yeast strains was confirmed by PCR as well as by replica plating on dropout plates. Yeast cells were cultured in standard YP growth media including YPD (1% yeast extract, 2% peptone and 2% glucose), YPGal (2% galactose), YPGE (3% glycerol+1% ethanol), or synthetic media (SC glucose). For qualitative growth measurement, 10-fold serial dilutions of overnight cultures were spotted on YPD or YPGE plates and incubated at 30° C. and 37° C. for the indicated period. Growth in liquid media was measured spectrophotometrically at 600 nm.

TABLE 2

| Strain | Genotype |
|---|---|
| BY4741 WT | MAT a, his3Δ1, leu2Δ0, met15Δ0, ura3Δ0 |
| BY4741 coa6Δ | MAT a, his3Δ1, leu2Δ0, met15Δ0, ura3Δ0, |
| | coa6Δ:: KanMX4 |
| BY4741 sco1Δ | MAT a, his3Δ1, leu2Δ0, met15Δ0, ura3Δ0, |
| | sco1Δ:: KanMX4 |
| BY4741 sco2Δ | MAT a, his3Δ1, leu2Δ0, met15Δ0, ura3Δ0, |
| | sco2Δ:: KanMX4 |
| BY4741 cox12Δ | MAT a, his3Δ1, leu2Δ0, met15Δ0, ura3Δ0, |
| | cox12Δ:: KanMX4 |
| BY4741 ctr1Δ | MAT a, his3Δ1, leu2Δ0, met15Δ0, ura3Δ0, |
| | ctr1Δ:: KanMX4 |
| BY4741 atx1Δ | MAT a, his3Δ1, leu2Δ0, met15Δ0, ura3Δ0, |
| | atx1Δ:: KanMX4 |
| BY4741 ccs1Δ | MAT a, his3Δ1, leu2Δ0, met15Δ0, ura3Δ0, |
| | ccs1Δ:: KanMX4 |
| BY4741 gsh1Δ | MAT a, his3Δ1, leu2Δ0, met15Δ0, ura3Δ0, |
| | gsh1Δ:: KanMX4 |
| BY4741 ccc2Δ | MAT a, his3Δ1, leu2Δ0, met15Δ0, ura3Δ0, |
| | ccc2Δ:: KanMX4 |
| BY4741 gef1Δ | MAT a, his3Δ1, leu2Δ0, met15Δ0, ura3Δ0, |
| | gef1Δ:: KanMX4 |
| BY4741 pic2Δ | MAT a, his3Δ1, leu2Δ0, met15Δ0, ura3Δ0, |
| | pic2Δ:: KanMX4 |
| STY11 pic2Δcoa6Δ | MAT a, his3Δ1, leu2Δ0, met15Δ0, ura3Δ0, |
| | pic2Δ:: KanMX4, coa6Δ:: NatMX4 |

Mammalian Cell Culture. The human control MCH46 and SCO2 patient fibroblasts as well as the rat H9c2 control and Ctr1^{-/-} cardiomyocytes were cultured in high glucose Dulbecco's modified Eagle's medium (DMEM) supplemented with 10% fetal bovine serum (Sigma) and 1 mM sodium pyruvate (Life Technologies). The mouse embryonic fibroblasts were cultured in DMEM 10% FBS, 1 mM sodium pyruvate, 1× minimum essential medium non-essential amino acids (MEM NEAA; Life Technologies 11140), 50 μg/mL uridine, and 1× Pen Strep Glutamine (Life Technologies 10378). All cell lines were cultured under 5% CO₂ at 37° C. and were treated with indicated concentrations of ES for 3-6 days before harvesting. Whole cell protein was extracted in lysis buffer (BP-115, Boston BioProducts) supplemented with protease inhibitor cocktail (Roche Diagnostics) and the protein concentrations were determined by BCA assay (Thermo Scientific).

Construction of a Ctr1 Knockout Rat H9c2 Cell Line. A CRISPR/Cas9 mediated Ctr1 knockout rat H9c2 cell line was generated by using lentiCRISPR v2 plasmid (Addgene, #52961). A guide RNA (gRNA) sequence targeting exon 1 of the Ctr1 gene was identified using the online CRISPR design tool. Forward (5′ CACCGTGGTGATGTTGTCGTCCGTG 3′) (SEQ ID NO: 7) and reverse (5′ AAACCACGGACGACAACATCACCAC 3′) (SEQ ID NO: 8) oligonucleotides were inserted into lentiCRISPR v2 plasmid. The transfection was performed using PolyJet (SignaGen Laboratories). Two days after transfection, cells were plated on a 96-well plate containing 5 μg/mL puromycin selection media. Each colony formed from single cells was isolated and established in medium without puromycin. Disruption of the Ctr1 gene was confirmed by genomic DNA sequencing.

Oxygen Consumption Measurement. For measurements of respiration rates, cells were grown to late log phase in YPGal medium and then washed, counted, and resuspended in fresh YPGal medium at 10⁸ cells/ml. The rate of oxygen consumption was then measured at 30° C. using the Oxytherm (Hansatech, Norfolk, UK). Cyanide-sensitive respiration was calculated after the addition of 1 mM KCN, and the cyanide-insensitive respiration was subtracted from the total respiration.

Immunoblotting and In-gel Activities. Sodium dodecyl sulfate polyacrylamide gel electrophoresis (SDS-PAGE) and Blue Native PAGE (BN-PAGE) were performed to separate denatured and native protein complexes, respectively. For SDS-PAGE, mitochondrial lysate (20 g) was separated on NuPAGE 4-12% Bis-Tris gels (Life Technologies, Carlsbad, CA). For BN-PAGE, yeast mitochondria were solubilized in buffer containing 1% digitonin (Life Technologies) by incubating for 15 min at 4° C. Clear supernatant was collected after a 20,000×g (30 min, 4° C.) spin, 50×G-250 sample additive was added, and 20 μg of protein was loaded on a 3-12% native PAGE Bis-Tris gel (Life Technologies). Following wet transfer, the membrane was probed with the following primary antibodies: for yeast proteins—Cox2, 1:50,000 (110 271; Abcam) and porin, 1:50,000 (110 326; Abcam), for mammalian proteins—COX1 (14705; Abcam), COX2 (110258; Abcam), CTR1, COX4 (A21348; Thermo Fisher Scientific), CCS (FL-274; Santa Cruz Biotechnology), GAPDH (G9545, Sigma), ATP5A (14748; Abcam), and 3-actin (A2228; Sigma). Western blots were developed using Western Lightning Plus-ECL (PerkinElmer, Waltham, MA). In-gel activity assay for mitochondrial respiratory chain complex IV was performed.

Cellular and Mitochondrial Copper Measurements. Cellular and mitochondrial copper levels were measured using the Perkin Elmer DRC II Inductively Coupled Plasma-Mass Spectrometer (ICP-MS). Intact yeast cells and isolated mitochondrial pellets were washed with 100 μM EDTA containing water, weighed, and digested with 40% nitric acid (TraceSELECT, Sigma) at 90° C. for 18 h. Samples were diluted in ultrapure metal-free water (TraceSELECT, Sigma) and analyzed by ICP-MS. Copper standard solutions were prepared by appropriate dilutions of commercially available mixed metal standards (BDH Aristar Plus). Copper concentrations in mammalian cells were also measured by ICP-MS.

Zebrafish Experiments. Zebrafish studies were approved by the Marine Biological Laboratory Institutional Animal Care and Use Committee (#16-38). Wild type AB strain and Ctr1 heterozygous zebrafish were maintained and crossed using standard methods. Embryos were staged and raised in Egg Water at 28.5° C. For drug treatments, embryos from Ctr1 heterozygous crosses were incubated in 10 nM ES diluted in Egg Water beginning at 3 hours post-fertilization (hpf). For imaging live embryos at 48 hpf, representative embryos of each sample were anesthetized in Tricaine and imaging was performed on an Olympus SZX12 stereomicroscope. For immunoblots, zebrafish mitochondrial protein was prepared from 10 days post-fertilization (dpf) larvae. Mitochondrial lysate was separated by SDS-PAGE on 4-15% Mini-PROTEAN TGX Gels (Bio-Rad) followed by Western blot analysis using anti-Cox1 at 1:5000 (anti-MTCO1; Abcam; ab14705) and anti-Atp5a at 1:5000 (Abcam; ab110273). Morpholino-based experiments were performed.

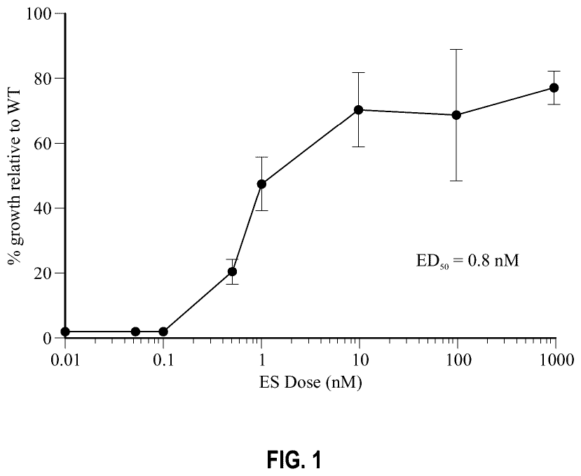

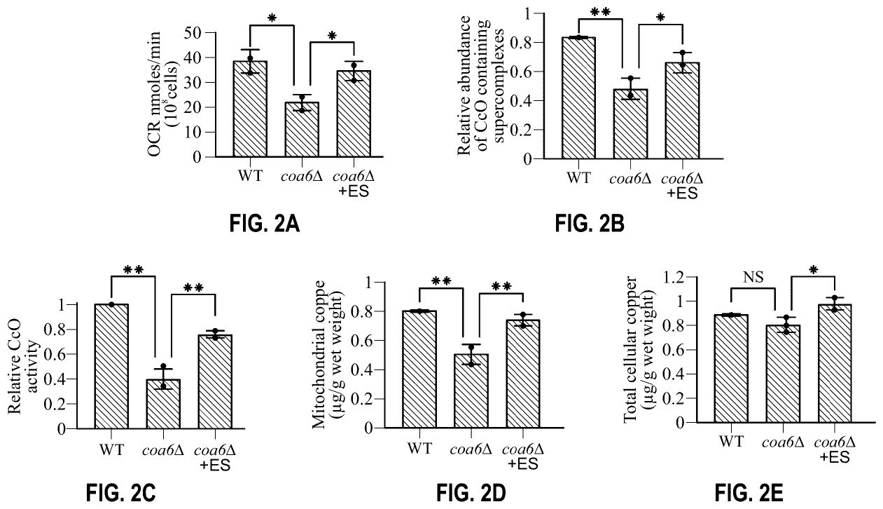

A Targeted Search for Copper-binding Agents Identifies Elesclomol as the Most Potent Pharmacological Agent in Rescuing Respiratory Defects of Yeast coa6Δ Cells. A number of copper-binding pharmacological agents were tested for their ability to rescue respiratory deficient growth of coa6Δ cells. Among all the compounds tested, ES was unique in that it rescued respiratory growth at low nanomolar concentrations without exhibiting overt toxicity over a broad range of concentrations. ES rescued the respiratory growth of coa6Δ cells with an ED₅₀ of 0.8 nM (FIG. 1). ES-mediated growth rescue of coa6Δ cells was also observed on solid growth medium containing a non-fermentable carbon. Consistent with the rescue of respiratory growth, ES supplementation restored the oxygen consumption rate of coa6Δ cells to that of wild type cells (FIG. 2A). To determine the biochemical basis for the observed respiratory rescue of coa6Δ cells, the assembly and activity of CcO-containing mitochondrial respiratory chain supercomplexes was measured by native-polyacrylamide gel electrophoresis blotting and in-gel activity assay, respectively. ES supplementation restored the abundance and activity of CcO-containing supercomplexes to near wild type levels (FIG. 2B-FIG. 2C). Next, the efficacy of ES in rescuing COA6 mutations observed in human patients was tested by heterologous expression of yeast-human chimeric proteins with patient mutations (W26C, W33R and E54X) in yeast coa6Δ cells. Similar to coa6Δ cells, 10 nM ES or 10 μM copper supplementation rescued yeast coa6Δ cells expressing patient mutations. These results show that ES is at least 1000 times more potent than copper in rescuing the respiratory growth defect of yeast coa6Δ cells.

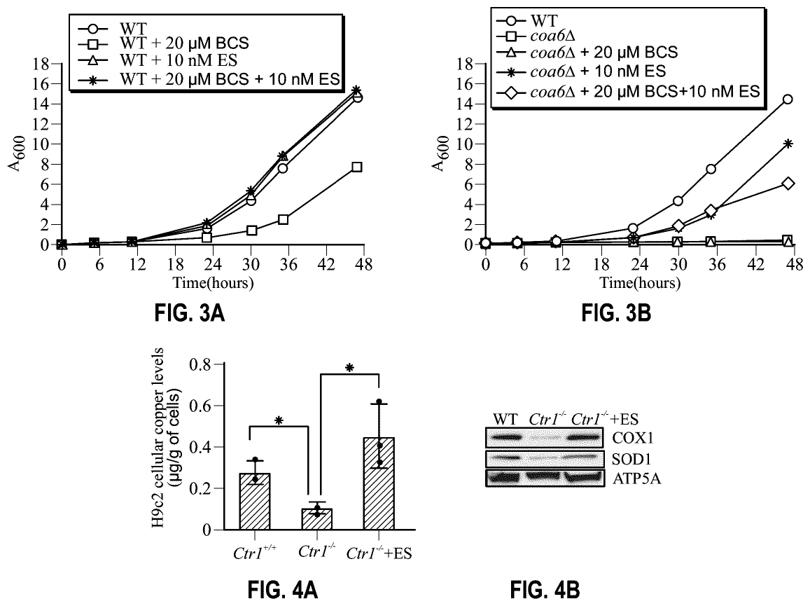

ES scavenges copper from the culture medium, enters the cell as an ES-copper complex, and selectively accumulates in mitochondria where it dissociates from copper. Consistent with this concept, an almost complete rescue of mitochondrial copper levels in coa6Δ cells supplemented with ES was observed (FIG. 2D). ES supplementation also moderately increased total cellular copper levels (FIG. 2E). To further corroborate that ES increases mitochondrial copper levels by actively transporting extracellular copper into the cells, copper availability in the extracellular compartment was decreased by co-treatment of ES with a known copper chelator, bathocuproine disulfonate (BCS). As expected, BCS treatment resulted in reduced respiratory growth of wild type cells, which was rescued by co-treatment with ES, suggesting that ES is also able to overcome pharmacological copper deficiency by outcompeting BCS (FIG. 3A). Moreover, ES mediated rescue of coa6Δ was diminished in the presence of BCS (FIG. 3B). To determine whether ES is able to bypass a mitochondrial copper transporter, Pic2, ES supplementation in pic2Δ and coa6Δpic2Δ cells was performed. Although under the conditions tested a respiratory growth defect of pic2Δ cells was not observed, ES did rescue coa6Δpic2Δ cells, suggesting that this compound can deliver copper to the mitochondria independent of Pic2 function. These results imply that ES mediated rescue of CcO function is driven by its ability to transport extracellular copper to the mitochondria of coa6Δ cells.

ES Rescues Many Different Yeast Mutants with Impaired Copper Metabolism. To test the specificity of ES mediated rescue, a number of yeast mutants of genes required for maintaining cellular and mitochondrial copper homeostasis were shortlisted. Genes were prioritized based on their evolutionary conservation, presence of pathogenic mutations in humans, and/or the existence of a related mouse phenotype (Table 3, shown below). These yeast mutants showed a pronounced respiratory deficient growth phenotype in non-fermentable media at 37° C. after two days of growth, which became less evident after four days of growth. Most of the yeast mutants were rescued with ES supplementation, albeit to different degrees, reflecting their distinct roles in cellular and mitochondrial copper homeostasis. ES failed to rescue sco1Δ cells, possibly because of the specific role of Sco1 as a metallochaperone in inserting copper into the Cox2 subunit of CcO. It was noticed that a higher concentration of ES is required to rescue ctr1Δ cells which is consistent with the severe reduction in copper levels in cells lacking Ctr1. Overall, these results suggest the broad applicability of ES in ameliorating defects of cellular and mitochondrial copper homeostasis.

TABLE 3

| Yeast | Human | | Clinical Phenotypes of Human |
|---|---|---|---|
| Gene | Gene | Role in Copper Metabolism | Disease or Animal Model (OMIM) |
| COA6 | COA6 | Required for copper delivery to | Fatal infantile |
| | | COX2 | cardioencephalomyopathy (614772) |
| SCO1 | SCO1 | Transfer of copper to Cu_{A} site | Neonatal hepatopathy and hypertrophic |
| | | in COX2 | cardiomyopathy (603644) |
| SCO2 | SCO2 | Oxidoreductase required for | Neonatal encephalo-cardiomyopathy |
| | | copper transfer to COX2 | (604272) |
| COX12 | COX6B1 | Cytochrome c oxidase subunit | Encephalomyopathy and hypertrophic |
| | | | cardiomyopathy (124089) |
| CTR1 | CTR1 | High-affinity copper | Ctrl^{-/-} mice are embryonic lethal |
| | | transporter | (603085) |
| ATX1 | ATOX1 | Delivery of cytosolic copper to | Failure to thrive and increased perinatal |
| | | ATP7A and ATP7B | mortality in Atox1-null mice (602270) |
| CCS1 | CCS | Copper delivery to Cu/Zn SOD | Congenital cataracts, hearing loss, and |
| | | | neurodegeneration (603864) |
| GSH1 | GSX1 | Catalyzes the first step in | Growth retardation and infertility in |
| | | glutathione biosynthesis | Gsh1-null mice (616542) |
| CCC2 | ATP7A | Copper transporting P-type | Menkes disease, occipital horn syndrome |
| | | | and spinal muscular atrophy (300011) |
| | ATP7B | ATPase | Wilson disease - liver disease and |
| | | | neurological defects (606882) |
| GEF1 | CLCN5 | Voltage-gated chloride channel | Dent disease - chronic kidney failure |
| | | | (300008) |
| | CLCN7 | | Osteopetrosis - bone defect (602727) |

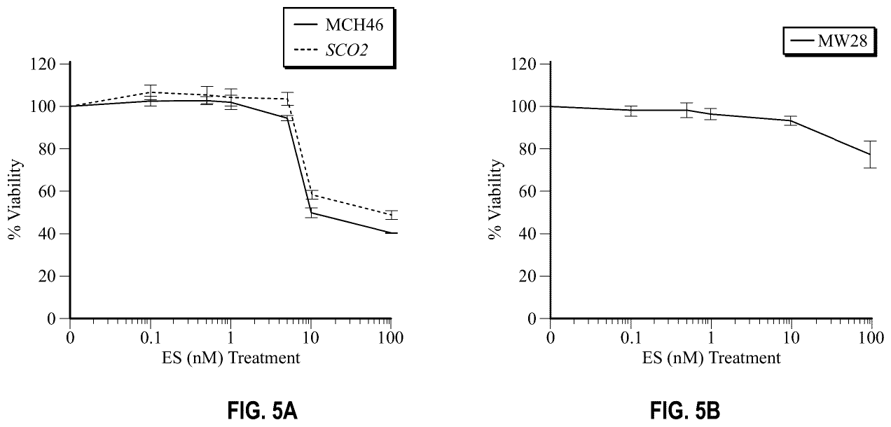

ES Supplementation Rescues Levels of CcO Subunits in Mammalian Cell Lines with Genetic Defects in Copper Metabolism. To expand upon the findings in yeast and to test the efficacy of ES in mammalian cell culture models of copper deficiency, a Ctr1 knockout rat H9c2 cardiomyocyte cell line was constructed. The Ctr1^{-/-} cell line was validated by demonstrating the loss of Ctr1 protein. As expected, the loss of Ctr1 led to a ˜4-fold decrease in the levels of intracellular copper (FIG. 4A) and a concomitant reduction in the levels of the CcO subunitCOX1 and superoxide dismutase, SOD1 (FIG. 4B). This validated Ctr1^{-/-} cell line was used to test the efficacy of ES in rescuing COX1, a copper-containing subunit of CcO and SOD1, superoxide dismutase, another copper-containing enzyme SOD1. As shown in FIG. 4A, Ctr1^{-/-} cells display reduced copper levels, which are restored by supplementation with 5 nM ES. FIG. 4B shows that 5 nM ES treatment of Ctr1^{-/-} cells also restores the levels of COX1 and SOD1. Similarly, a dose dependent rescue of COX1 levels in Ctrl^{-/-} mouse embryonic fibroblasts (MEFs) was observed. However, ES toxicity was noticed at higher doses, which was reflected in reduced COX1 levels in control MEFs. Finally, the efficacy of ES in rescuing CcO defects in SCO2 patient fibroblasts that were previously shown to have a COX2 deficiency was tested. Unlike copper supplementation, ES treatment was able to partially rescue the steady state levels of COX2 in a dose and time dependent manner. Notably, ES toxicity occurs at a concentration 20× higher than the dose that rescued COX2 levels (FIG. 5A). Consistent with the known anti-carcinogenic activity of ES, much more pronounced toxicity in immortalized cell lines (FIG. 5A) when compared to primary cultures of the same cell type (FIG. 5B) was observed. Together, these results suggest that a low nanomolar concentration of ES is efficacious in rescuing mitochondrial copper deficiency and restoring CcO levels in mammalian cell lines with genetic defects in copper homeostasis.

ES Supplementation Rescues Copper Deficiency Phenotypes in Zebrafish Models. To determine whether ES can rescue phenotypes associated with copper deficiency in an intact developing vertebrate animal model, zebrafish embryos with a null mutation in the gene encoding the plasma membrane copper importer Ctr1 were utilized. Zebrafish were chosen because of the ability to quickly monitor the pigmentation defect that arises due to the copper requirement of tyrosinase, an enzyme that catalyzes the critical step in melanin biosynthesis. Wild type zebrafish embryos have a characteristic melanin pigmentation pattern visible at 48 hpf. To determine if ES can rescue copper deficiency phenotypes in zebrafish, zebrafish embryos from heterozygous Ctr1 crosses in 10 nM ES were incubated and compared to untreated embryos. It was found that the expected ˜25% of untreated embryos from Ctr1 heterozygous crosses lacked melanin deposition, whereas all of the ES treated embryos from the same crosses were pigmented. Similarly, rescue of the pigmentation defect at 100 nM ES was observed, but the equivalent dose of copper failed to rescue this defect. Ctr1^{-/-} mutants also exhibited a CcO assembly defect likely due to mitochondrial copper deficiency. To determine whether ES can rescue the observed CcO assembly defect, clutches of embryos were grown from heterozygous Ctr1 crosses in the presence of ES until 10 dpf and levels of Cox1 in their mitochondrial extracts were measured. Compared to wild type embryos, the Ctrl1^{-/-} mutants exhibited a severe reduction in Cox1 levels that was almost completely rescued by treatment with ES.

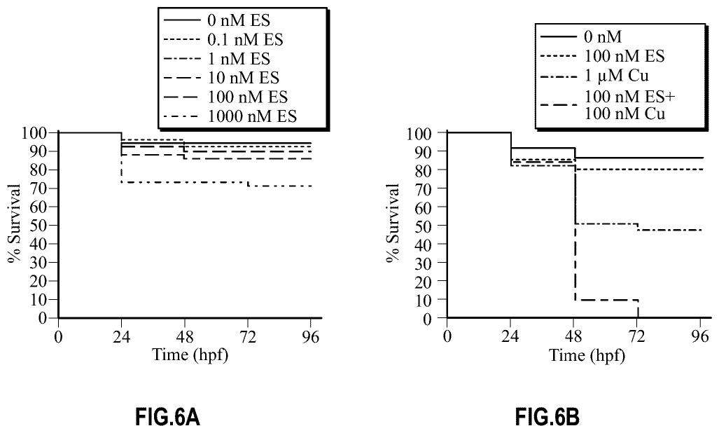

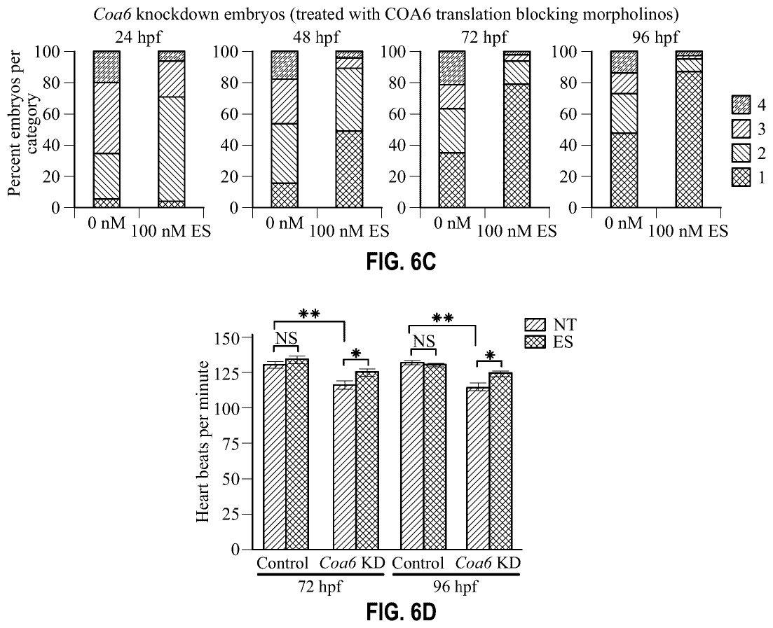

To further establish the potential of ES to treat metabolic diseases involving defective copper delivery to the mitochondrion, the efficacy of ES in rescuing phenotypes associated with Coa6 knockdown in zebrafish embryos was tested. First, the maximal tolerable dose of ES for zebrafish embryos was determined to be 100 nM (FIG. 6A). Consistent with the mechanism of action of ES, it was observed that co-supplementation of 100 nM ES with 100 nM of copper resulted in 100% lethality (FIG. 6B). Zebrafish embryos injected with the zfcoa6 translation blocking morpholino exhibited pronounced morphological defects characterized by pericardial edema, smaller heads and eyes, and curved tails. The severity of these phenotypes was scored at four different time points, 24, 48, 72, and 96 hpf, illustrated in Table 4 below, and rescue with 100 nM ES treatment was observed as early as 48 hpf (FIG. 6C). Given that one of the most striking features of Coa6 deficiency in this model is a pronounced cardiac edema and a decreased heart rate, next it was determined whether ES treatment was able to rescue these phenotypes. Indeed, 100 nM ES treatment prevented pericardial edema and significantly increased the heart rate of Coa6 knockdown zebrafish embryos at both 72 and 96 hpf, without altering the heart rate of control embryos (FIG. 6D). These results demonstrate the efficacy of ES in rescuing phenotypes associated with copper deficiency in intact living vertebrate animals.

TABLE 4

| Phenotype Score | 1 | 2 | 3 | 4 |
|---|---|---|---|---|
| 24 hpf | normal | slightly smaller head, | smaller head/eyes, | very small head/eyes, |
| | | normal tail | slight graininess in | very short tail kinked |
| | | | head, shorter tail | at end |
| 48 hpf | normal | smaller head/eyes, | smaller head/eyes, | smaller head/eyes, |
| | | slight PE and normal | PE, shortened tail | PE, tail kinked at end |
| | | tail tip | | or shortened at YSE |
| 72 hpf | normal | smaller head/eyes, | smaller head/eyes, | smaller head/eyes, |
| | | slight PE and normal | PE, shortened tail | PE, tail kinked at end |
| | | tail tip | | or shortened at YSE |
| 96 hpf | normal | smaller head and | small head and eyes, | very small head and |
| | | eyes, tail normal but | tail tip clubbed or | eyes, eyes misshapen, |
| | | shorter, PE | kinked down, tail | little to no tail past |
| | | | shorter, PE | YSE, PE |

PE = pericardial edema;
YSE = yolk sac edema

Mitochondrial disorders of copper metabolism represent a subset of inborn errors of mitochondrial energy metabolism for which no therapy currently exists. A previous attempt to use direct copper supplementation as a therapeutic approach was unsuccessful, possibly due to stringent regulation of systemic copper levels. Thus, there is an unmet need for developing better copper delivery agents. With this goal in mind, a number of clinically used pharmacological agents were tested on a yeast mitochondrial disease model of COA6 deficiency and identified ES as the most potent and best-tolerated compound capable of restoring mitochondrial function. Subsequent experiments on other yeast, murine, human, and zebrafish models established broad applicability of ES in treating mitochondrial, cellular and organismal copper deficiency. ES has undergone multiple human clinical trials where it has exhibited a favorable toxicity profile. Thus, the findings presented herein offer the possibility of repurposing this anti-cancer drug for the treatment of disorders of copper metabolism.

Pharmacological interventions that alter the subcellular concentration and distribution of metals in a targeted manner could be of therapeutic benefit. For example, co-administration of copper with disulfiram, a Food and Drug Administration approved drug, increased the activity of CcO in the brains of a mouse model of Menkes disease, a genetic disorder characterized by systemic copper deficiency. Similarly, Cu^{II}-ATSM has been shown to be efficacious in a transgenic mouse model of amyotrophic lateral sclerosis. However, a comparative study on the efficacy of these clinically used copper complexes in a model of copper deficiency is lacking. Therefore, the present disclosure, identifying ES as the most potent pharmacological agent among many of the clinically used copper chelators and ionophores, represents an important advancement. The physicochemical properties of ES, including its binding affinity, its specificity for copper, and the redox potential of the ES-copper complex, allow it to mimic a copper metallochaperone. Higher affinity of ES for copper (II) compared to copper (I) allows it to scavenge copper from the extracellular environment where copper is more likely to exist in an oxidized state. ES is unlikely to strip copper from intracellular proteins, because of the higher prevalence of copper in the reduced state in the intracellular environment.

While there is selective enrichment of the ES-copper complex in the mitochondria, the rescue of yeast atx1Δ and ccc2Δ, which have impaired copper homeostasis in the Golgi compartment, suggests that ES is also able to deliver copper to other subcellular compartments. Indeed, the rescue of the pigmentation defect observed in Ctr1^{-/-} zebrafish caused by a defective secretory pathway enzyme also indicates that ES could increase copper levels in other organelles. Finally, the rescue of the respiratory growth defect of ccs1Δ cells, which are deficient in a metallochaperone for the cytosolic protein Sod1, suggests that ES is also able to elevate cytosolic copper levels. While the mechanism by which ES is able to deliver copper to different subcellular compartments is not entirely clear, it is possible that some of the ES-copper complexes dissociate before reaching mitochondria, thereby releasing free copper in the cytoplasm. Alternatively, excess mitochondrial copper may “leak” out of the mitochondria and become available to other organelles. Notwithstanding the mechanism, this interesting observation suggests that ES could be efficacious in the treatment of more common disorders of copper deficiency, including Menkes disease, as further discussed below.

### Mouse Model of Human Copper Deficiency

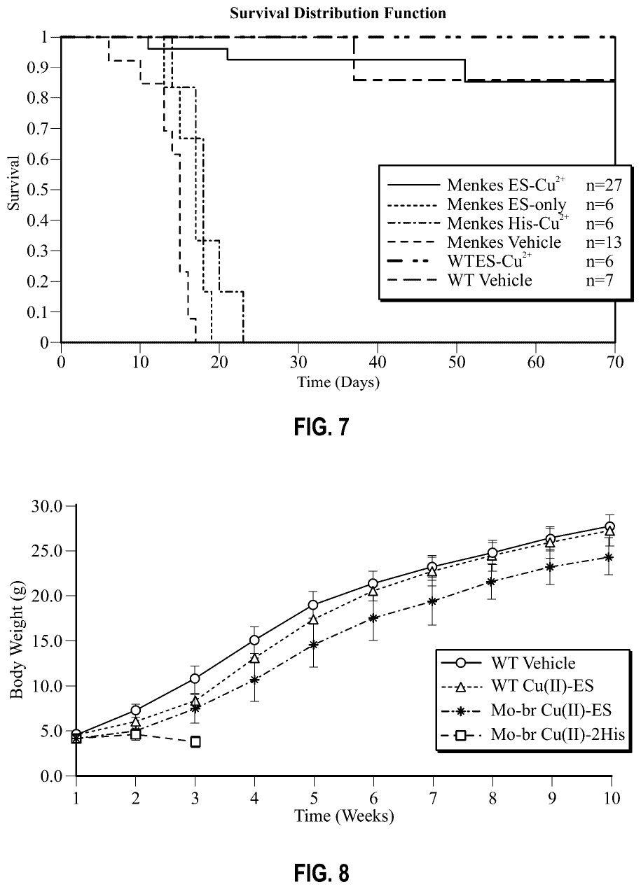

In Vivo Mouse Model Data Demonstrating the Efficacy of Cu-Elesclomol Complex [Cu(II)-ES]. To evaluate the potential of Cu(II)-ES in a mouse model of human copper deficiency disorder, the well-characterized mottled-brindled (mo-br) mouse model (C57BL/6-Atp7a^{mo-br}/J, Jackson Laboratory Stock #002566) that closely mimics the clinical and biochemical phenotypes of Menkes disease was chosen. Like in humans, these mice are characterized by mutation in ATP7a, which is an X-linked gene and therefore its mutation only affects males. Importantly, as with human Menkes patients, the mo-br mice cannot be rescued by the injection of Cu salts. Menkes-affected mice typically die by approximately day 14, and at postnatal day 10 they start showing neurological defects, for example, seizures, loss of righting reflex, and the like. Administering two subcutaneous doses of 3.625 mg/kg/dose of Cu(II)-ES on postnatal day 7 and 10 was sufficient to rescue the mo-br mice from death (FIG. 7). Notably, the vehicle, copper-histidinate or the elesclomol alone treated mo-br mice died within 20 days of birth, whereas 81.5% of Cu(II)-ES treated mo-br mice were surviving until day 70 (FIG. 7). All wild type treated with either vehicle or Cu(II)-ES survived to day 70 without adverse effects suggesting that the current dosing and treatment regimen is not toxic to the mice.

To test whether Cu(II)-ES treatment of mo-br mice improves only the survival or also promote growth, weight gain following two injections of Cu(II)-ES was monitored. Mo-br mice treated with Cu(II)-ES demonstrated weight gain pattern similar to that of wild type mice, however the weight gain was slightly lower at all the assessed time points (P<0.001), as shown in FIG. 8. Mild growth deficiencies were noticed in wild type mice treated with Cu(II)-ES when compared to wild type vehicle treated mice at weeks 2, 3, and 4 of assessment (P=0.0004, 0.0003, and 0.026), but these mice recovered by week 5 (P>0.05 weeks 5-10), as shown in FIG. 8. These data show that Cu(II)-ES treated mo-br mice not only survive but also thrived, and that Cu(II)-ES treatment does not have any long term growth retardation effect on wild type mice.

In addition to the survival and growth measurements, a number of neurological tests were performed to evaluate the efficacy of ES-mediated copper delivery to the brain and overall wellbeing of the animals. These tests, described in further detail below, were conducted on mice that were treated only twice (day 7 and day 10) as described previously.

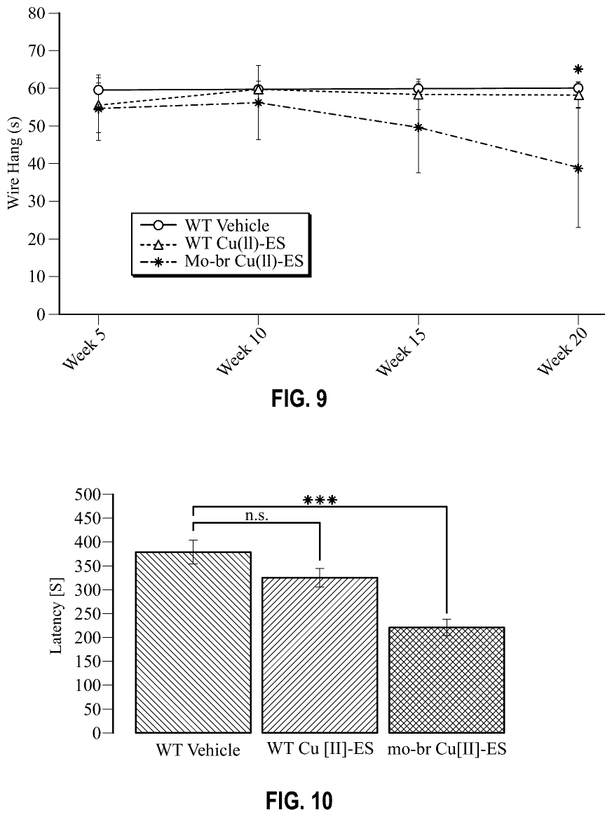

Wire Hang Test. This test evaluates motor function and muscle strength of animals. Wild type mice treated with either vehicle or Cu(II)-ES were indistinguishable with respect to their hang time, implying that the administration of Cu(II)-ES does not cause any muscle or motor function toxicity (FIG. 9). In terms of drug efficacy, Cu(II)-ES treated mo-br mice exhibit statistically similar hang time to that of wild type mice treated with vehicle until week 15, but there was a significant decline (*P=0.01) in muscle strength at week 20 (FIG. 9). This decline in muscle function after week 15 in mo-br mice suggests that copper levels might be declining after 15 weeks of life.

Rotarod Test. This test evaluates neuromotor function including balance, endurance, grip strength, and motor coordination. It involves placing adult mice on a horizontally oriented rotating rod suspended over the cage floor and monitoring the time until they fall. As shown in FIG. 10, mo-br mice rescued with Cu(II)-ES treatment demonstrated significantly lower latency to fall as compared to wild type mice treated with vehicle, with mean fall times of 222.0±34.5 seconds versus 379.0±51.5 seconds (***P<0.0001). Wild type mice treated with Cu(II)-ES showed no significant difference as compared to vehicle treated wild type mice with mean fall times of 326.3±37.8 seconds (P=0.182). This data suggest a partial rescue in neuromotor function with the current dosage and treatment regimen of ES-Cu (two injections each with 14.5 μg of ES-Cu at day 7 and day 10 after birth).

### Mouse Model of Human Copper Deficiency Results and Discussion

The in vivo mouse model data suggests that the Cu(II)-ES formulation could be efficacious in a number of human disorders characterized by dysregulation of copper metabolism. For example, Cu(II)-ES formulations could be efficacious for occipital horn syndrome and X-linked distal hereditary motor neuropathy, both of which are caused by mutations in ATPA7A gene and are “milder” versions of Menkes. Additionally, Cu(II)-ES formulations could be efficacious for amyotrophic lateral sclerosis or Lou Gehrig disease. Amyotrophic lateral sclerosis is caused by mutation in Cu/Zn-superoxide dismutase (SOD1) and augmenting copper delivery to SOD1 is therapeutically beneficial.

Moreover, Cu(II)-ES formulations could be efficacious for Alzheimer's disease. The salient feature of Alzheimer's disease is the accumulation of extracellular β-amyloid (A3) plaques in the brain. It has been shown that copper delivery by either diet or pharmacological means can reduce interstitial A3 and improve cognitive function in transgenic mouse models of Alzheimer's. Also, Cu(II)-ES formulations could be efficacious for Huppke-Brendel syndrome. This syndrome is caused by mutations in AT-1 gene that encodes the endoplasmic reticulum membrane acetyl-CoA transporter, which is required for acetylation of one or more copper proteins. Mutations in AT-1 result in lower serum copper levels and profound neurological defects, thus Cu(II)-ES could be therapeutically beneficial in this condition. Furthermore, Cu(II)-ES formulations could be efficacious for MEDNIK syndrome. This syndrome is caused by mutation in AP1S1 gene and is characterized by perturbation in copper metabolism with reduced expression of cytochrome c oxidase and SOD1, the copper dependent enzymes.

As such, it is envisioned that Cu(II)-ES formulations could be efficacious in a number of human disorders characterized by dysregulation of copper metabolism including, but not limited to, occipital horn syndrome, X-linked distal hereditary motor neuropathy, amyotrophic lateral sclerosis, Lou Gehrig disease, Alzheimer's disease, Huppke-Brendel syndrome, MEDNIK syndrome, or combinations of the same and like.

It has been demonstrated above that a total of two injections of Cu(II)-Elesclomol, each 3.635 mg/kg on postnatal day 7 and 10 administered via subcutaneous route is efficacious. This dose was chosen based on the expected whole body copper content of mice. A 7-day old 4 g pup is expected to have ˜4 μg of whole body copper. Thus, two doses of 3.635 mg/kg of Cu(II)-Elesclomol was chosen, with each dose containing 2 μg equivalent of copper for a total dose of 4 g. Based on these experiments, a dose range of 3 to 14.5 mg/kg of total Cu(II)-Elesclomol administered within the first 10 days of birth is likely to be effective in ameliorating Menkes-related phenotypes in mo-br mice.

In view of the foregoing, in some embodiments, the present disclosure further pertains to therapeutically effective dosses in human subjects with disorders characterized by dysregulation of copper metabolism. As such, human doses can be extrapolated based on the above data, and is readily envisioned to one skilled in the art.

For example, in some embodiments, the therapeutic dosage of elesclomol-Cu(II) in humans is approximately of around 0.589 mg/kg body weight. Thus, for a human child weighing 4 kg, the dose would be approximately 2.36 mg. In some embodiments, the therapeutic dosage range for elesclomol in humans is 0.243-1.17 mg/kg. The human dosages are calculated as per Nair and Jacob (2016) Journal of Basic and Clinical Chemistry, Vol. 7 (2): 27-31.

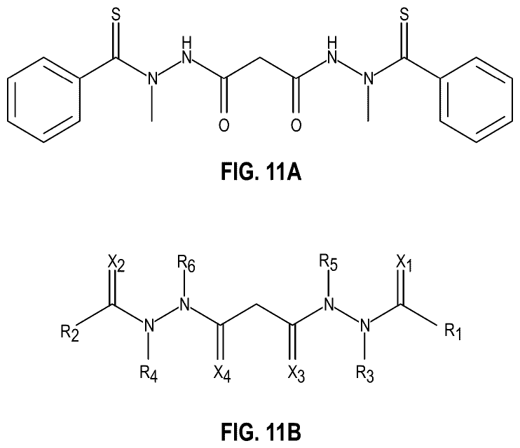

Although the formulation of elesclomol that has been shown to be most efficacious is Cu(II)-Elesclomol solubilized in 20% CAPTISOL® solution, other embodiments are readily envisioned. For example, in some embodiments, elesclomol analogs, mimetics, and derivatives thereof can be utilized as a substitute for elesclomol. FIG. 11A illustrates the chemical structure of elesclomol.#

In some embodiments, elesclomol analogs, mimetics, or derivatives can have the chemical structure as depicted in FIG. 11B. Referring to FIG. 11B, in some embodiments, X₁, X₂, X₃, and X₄ can each, independently, include O, S, Se, Te, Po, N(R₇)ₘ, P(R₇)ₘ, As(R₇)ₘ, Sb(R₇)ₘ, or Bi(R₇)ₘ, or combinations thereof. In some embodiments, m can include, without limitation, 0 or 1.

Still referring to FIG. 11B, in some embodiments, R₁, R₂, R₃, R₄, R₅, and R₆ can each, independently, include, —H, an alkyl, an alkenyl, an alkynyl, a cycloalkyl, a cycloalkenyl, a heterocyclyl, an aryl, a heteroaryl, a halogen, a nitro, a cyano, a guanadino, —OR₈, —NR₁₀R₁₁, —C(O)R₈, —C(O)OR₈, —OC(O)R₈, —C(O)NR₁₀R₁₁, —NR₉C(O)R₈, —OP(O)(OR₈)₂, —SP(O)(OR₈)₂, —SR₈, —S(O)ₚR₈, —OS(O)ₚR₈, S(O)ₚOR₈, —NR₉S(O)ₚR₈, —S(O)ₚNR₁₀R₁₁, an aromatic, or combinations thereof.

Still referring to FIG. 11B, in some embodiments, R₇ can each, independently, include —H, —OR₈, —NR₁₀R₁₁, —C(O)R₈, —C(O)OR₈, —OC(O)R₈, —C(O)NR₁₀R₁₁, —NR₉C(O)R₈, —OP(O)(OR₈)₂, —SP(O)(OR₈)₂, —SR₈, —S(O)ₚR₈, —OS(O)ₚR₈, —S(O)ₚOR₈, —NR₉S(O)ₚR₈, —S(O)ₚNR₁₀R₁₁, an alkyl, an alkenyl, an alkynyl, an cycloalkyl, an cycloalkenyl, an heterocyclyl, an aryl, a heteroaryl, an aralkyl, a heteraralkyl, a halogen, a nitro, a cyano, a guanadino, an aromatic, or combinations thereof. In some embodiments, p can include, without limitation, 1 or 2.

Still referring to FIG. 11B, in some embodiments, R₈, R₉, R₁₀, and R₁₁ can each, independently, include H, an alkyl, an alkenyl, an alkynyl, a cycloalkyl, a cycloalkenyl, a heterocyclyl, an aryl, a heteroaryl, an aralkyl, a heteraralkyl, a halogen, a nitro, a cyano, a guanadino, an aromatic, or combinations thereof. In some embodiments, R₁₀ and R₁₁ can each, independently, be taken together with the nitrogen to which they are attached to form a heterocyclyl or a heteroaryl.

In some embodiments, the compounds described above can be a pharmaceutical composition having a chemical structure as depicted in FIG. 11B, or a tautomer, pharmaceutically acceptable salt, solvate, clathrate, or prodrug thereof. In some embodiments, the compounds can further include a salt, solvent, buffer, diluents, binders, compression aids, granulating agents, disintegrants, glidants, lubricants, tablet coatings or films, coloring agents, or combinations thereof.

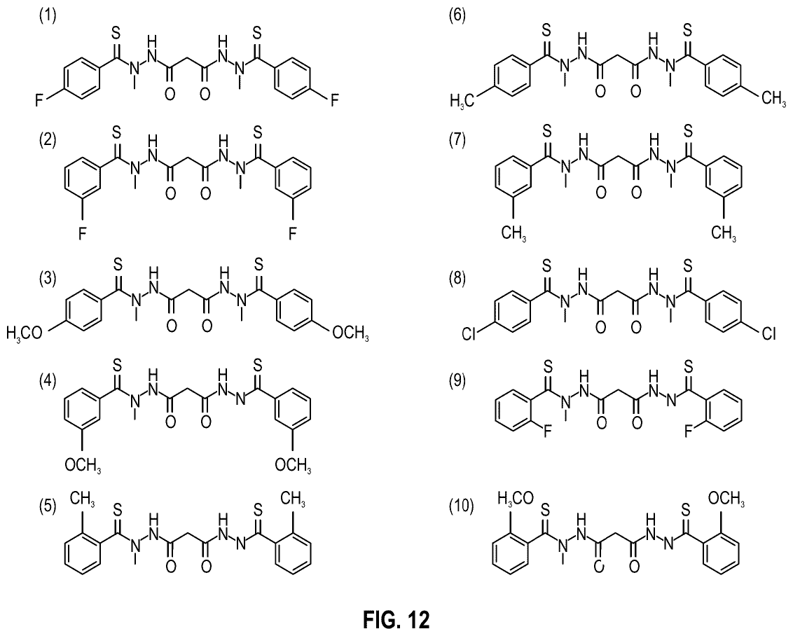

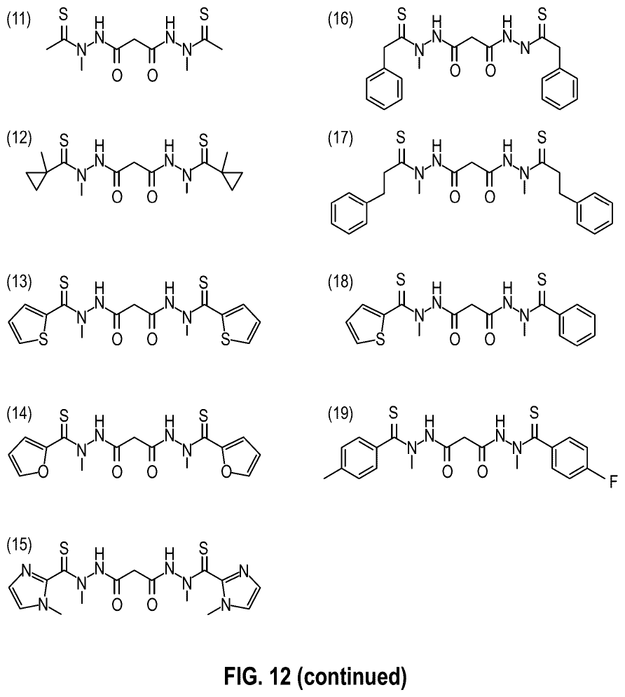

Further examples of chemical structures of elesclomol analogs, mimetics, and derivatives envisioned in the present disclosure are depicted in FIG. 12.

In view of the preceding, in some embodiments, the present disclosure relates to a method of restoring cytochrome c oxidase (CcO) activity in a cell. In some embodiments, the method includes contacting the cell with a therapeutically effective amount of elesclomol. In some embodiments, the contacting increases cellular and mitochondrial copper content. In some embodiments, the contacting reestablishes subcellular copper homeostasis in copper deficient cells. In some embodiments, the contacting rescues respiratory defects of cells deficient in COA6, SCO2, COX6B1, CTR1, ATOX1, CCS, GSX1, ATP7A, ATP7B, CLCN5 and CLCN7. In some embodiments, the contacting ameliorates defects of cellular and mitochondrial copper homeostasis. In some embodiments, the therapeutically effective amount of elesclomol can restore intracellular copper homeostasis by mimicking functions of missing transporters or chaperones of copper. In some embodiments, the therapeutically effective amount of elesclomol efficiently transports copper across biological membranes and restores mitochondrial respiratory chain function.

In additional embodiments, the present disclosure relates to a method of treating cellular or mitochondrial copper deficiency to a subject in need thereof. In some embodiments, the method includes administering a therapeutically effective amount of elesclomol. In further embodiments, the present disclosure relates to a method of rescuing CcO deficiency in fibroblasts from subjects with mutations in SCO2 and restores CcO activity in COA6 deficient yeast, where the method includes administering a therapeutically effective amount of elesclomol, where the elesclomol allows for efficient delivery of copper to mitochondria to restore CcO activity by bypassing SCO2 and COA6 functions.

In further embodiments, the present disclosure relate to a method of treating human disorders of copper metabolism. In some embodiments, the method includes administering a therapeutically effective amount of elesclomol either alone or in the presence of copper. In some embodiments, the disorder is caused by a mutation to the ATP7A gene. In some embodiments, the disorder is Menkes disease, occipital horn syndrome, or X-linked distal hereditary motor neuropathy.

Although various embodiments of the present disclosure has been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other methods and compositions for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an” and other singular terms are intended to include the plural forms thereof unless specifically excluded.

What is claimed is:

1. A method of treating disorders of copper metabolism, the method comprising:
administering a therapeutically effective amount of elesclomol to a subject;
wherein the disorder is caused by a deficiency or mutation to a gene selected from the group consisting of SOD1, AT-1, AP1S1, COA6, SCO2, COX6B1, CTR1, ATOX1, CCS, GSX1, ATP7A, ATP7B, CLCN5, CLCN7, or combinations thereof.

2. The method of claim 1, wherein the administering of elesclomol includes co-administering copper.

3. The method of claim 1, wherein the disorder is caused by a mutation to the ATP7A gene.

4. The method of claim 1, wherein the disorder is selected from the group consisting of occipital horn syndrome, X-linked distal hereditary motor neuropathy, amyotrophic lateral sclerosis, Lou Gehrig disease, Alzheimer's disease, Huppke-Brendel syndrome, MEDNIK syndrome, or combinations thereof.

5. The method of claim 1, wherein the elesclomol is an elesclomol analog, mimetic, or derivatives thereof.

6. The method of claim 1, comprising bypassing at least one of SCO2 functions and COA6 functions.

7. The method of claim 1, wherein the elesclomol is administered via injection.

8. The method of claim 2, wherein the elesclomol and copper are administered via injection.

9. The method of claim 1, wherein the elesclomol is complexed with copper.

10. The method of claim 2, wherein the elesclomol-copper complex is administered via injection.

11. A method of treating Menkes disease in a subject, the method comprising:
administering a therapeutically effective amount of elesclomol or a pharmaceutically acceptable salt thereof, in an amount effective to treat Menkes disease in the subject.

12. The method of claim 11, wherein the elesclomol is administered via injection.

13. The method of claim 11, wherein the administration further includes the co-administration of copper.

14. The method of claim 13, wherein the elesclomol and copper are administered via injection.

15. The method of claim 11, wherein the elesclomol is complexed with copper.

16. The method of claim 15, wherein the elesclomol-copper complex is administered via injection.

17. A method of treating Wilson's disease in a subject, the method comprising:
administering a therapeutically effective amount of elesclomol or a pharmaceutically acceptable salt thereof, in an amount effective to treat Wilson's disease in the subject.

18. The method of claim 17, wherein the elesclomol is administered via injection.

19. The method of claim 17, wherein the administration further includes the co-administration of copper.

20. The method of claim 19, wherein the elesclomol and copper are administered via injection.

21. The method of claim 17, wherein the elesclomol is complexed with copper.

22. The method of claim 21, wherein the elesclomol-copper complex is administered via injection.


SEQUENCE LISTING (8 sequences)

SEQ ID NO: 1 - DNA, 39 bp, synthetic construct
  misc_feature 1..39: note = HyCOA6 W26C Forward
  source 1..39: mol_type = other DNA
agtattcatctctagcaccgcaacacaattttctttggg

SEQ ID NO: 2 - DNA, 39 bp, synthetic construct
  misc_feature 1..39: note = HyCOA6 W26C Reverse
  source 1..39: mol_type = other DNA
cccaaagaaaattgtgttgcggtgctagagatgaatact

SEQ ID NO: 3 - DNA, 39 bp, synthetic construct
  misc_feature 1..39: note = HyCOA6 W33R Forward
  source 1..39: mol_type = other DNA
ttttcgtccaaacatttccggtattcatctctagcaccc

SEQ ID NO: 4 - DNA, 39 bp, synthetic construct
  misc_feature 1..39: note = HyCOA6 W33R Reverse
  source 1..39: mol_type = other DNA
gggtgctagagatgaataccggaaatgtttggacgaaaa

SEQ ID NO: 5 - DNA, 45 bp, synthetic construct
  misc_feature 1..45: note = HyCOA6 E54X Forward
  source 1..45: mol_type = other DNA
cattgttgtggacaagaagattagaaagaggatctcaacttctta

SEQ ID NO: 6 - DNA, 45 bp, synthetic construct
  misc_feature 1..45: note = HyCOA6 E54X Reverse
  source 1..45: mol_type = other DNA
taagaagttgagatcctctttctaatcttcttgtccacaacaatg

SEQ ID NO: 7 - DNA, 25 bp, synthetic construct
  misc_feature 1..25: note = Forward guide targeting exon 1 of the Ctr1 gene
  source 1..25: mol_type = other DNA
caccgtggtgatgttgtcgtccgtg

SEQ ID NO: 8 - DNA, 25 bp, synthetic construct
  misc_feature 1..25: note = Reverse guide targeting exon 1 of the Ctr1 gene
  source 1..25: mol_type = other DNA
aaaccacggacgacaacatcaccac